(19) United States
(12) Patent Application Publication
(10) Pub. No.: US 2025/0194978 A1
(43) Pub. Date: Jun. 19, 2025

(54) LARGE LANGUAGE MODEL WITH BRAIN PROCESSING TOOLS

(71) Applicant: Omniscient Neurotechnology Pty Limited, Sydney (AU)

(72) Inventors: Stephane Philippe Doyen, Mount Kuring Gai (AU); Michael Edward Sughrue, Brighton Le Sands (AU)

(21) Appl. No.: 18/540,775

(22) Filed: Dec. 14, 2023

(51) Int. Cl. A61B 5/16; A61B 5/00; G06F 40/40; G06T 7/00; G16H 20/70; G16H 40/67; G16H 50/20

(52) U.S. Cl. CPC A61B 5/165; A61B 5/4803; G06F 40/40; G06T 7/0012; G16H 20/70; G16H 40/67; G16H 50/20; G06T 2200/24; G06T 2207/10088; G06T 2207/30016

(57) ABSTRACT

Methods, systems, and apparatus, including computer programs encoded on computer storage media, for determining a mental state of a patient based on a natural language input and determining whether a relevant subset of brain data is anomalous. One of the methods includes receiving a natural language input describing at least one aspect of a mental state or of a behavior of an individual; generating a prompt based on at least in part the natural language input; submitting the prompt to a large language model; receiving at least one functional network that influences the at least one aspect of a mental state or of a behavior; for each network of the at least one functional network, analyzing MRI data for the individual to determine whether the network is anomalous; displaying to a user each network and whether it is anomalous; and taking an action in response to the displaying.

### BACKGROUND

### Technical Field

This specification relates to processing data related to a subject's brain using large language models. The present invention also relates to a method and apparatus for processing data related to a subject's brain, and to a computer program product including a computer readable medium having recorded thereon a computer program for processing data related to a subject's brain.

### Background

Brain functional connectivity data characterizes, for each of one or more pairs of locations within the brain of a patient, the degree to which brain activity in the pair of locations is correlated.

One can gather data related to the brain of the patient by obtaining and processing images of the brain of the patient, e.g., using magnetic resonance imaging (MRI).

Data related to the brain of a single patient can be highly complex and high-dimensional, and therefore difficult for a clinician to manually inspect and parse, e.g., to plan a surgery or diagnose the patient for a brain disease or mental disorder.

Large language models (LLMs) are a type of machine learning model that can be trained to receive a natural human language query and generate text and/or images in response to the query.

### SUMMARY

This specification describes technologies for determining a mental state or behavior of a patient that is of interest based on a natural language input and then determining whether a relevant subset of brain data is anomalous. These technologies generally involve processing a natural language input that describes aspects of the mental state or behavior of a patient using a large language model to identify and display regions of the patient's brain that may have abnormal activity levels.

In general, one innovative aspect of the subject matter described in this specification can be embodied in methods that include the actions of receiving a natural language input describing at least one aspect of a mental state or of a behavior of an individual; generating a prompt based at least in part the natural language input, the prompt configured to generate at least one functional network that influences the at least one aspect of a mental state or of a behavior above a threshold; submitting the prompt to a large language model; receiving an indication of at least one functional network from the large language model that influences the at least one aspect of a mental state or of a behavior; for each network of the at least one functional network, analyzing MRI data for the individual to determine whether the network is anomalous; displaying to the user each network of the at least one functional network and whether it is anomalous; and taking an action in response to the displaying. At least one aspect of a mental state or of a behavior can include more than one aspect of mental state(s), more than one aspect of behavior(s), or a combination of aspect(s) of mental state(s) and aspect(s) of behavior(s).

Other embodiments of this aspect include corresponding computer systems, apparatus, and computer programs recorded on one or more computer storage devices, each configured to perform the actions of the methods. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

The foregoing and other embodiments can each optionally include one or more of the following features, alone or in combination. In particular, one embodiment includes all the following features in combination.

In some implementations, the method further comprises receiving a user input in response to the displaying, wherein the response includes selection of at least one functional network to produce at least one selected functional network; and taking an action comprises submitting to a large language model a request for how to address an anomaly in the at least one selected functional network.

In some implementations, taking an action further comprises generating a plurality of treatment plans for how to address the anomaly in the at least one selected functional network.

In some implementations, a treatment plan comprises (i) surgeries, (ii) behavioral therapies, (iii) brain editing techniques, or a combination thereof.

In some implementations, the natural language input comprises one or more questions.

In some implementations, the method further comprises in response to submitting the prompt to the large language model, receiving a mental state or a behavior.

In some implementations, the method further comprises displaying the mental state or behavior along with an indication of whether that mental state or behavior is anomalous.

The subject matter described in this specification can be implemented in particular embodiments so as to realize one or more of the following advantages. A set of brain data characterizing the brain of a single patient can often be incredibly large and complicated, and thus it can be difficult and time consuming for a user to extract useful information from the set of brain data. Using techniques described in this specification, a system can quickly identify one or more networks in a patient's brain that may have abnormal activity and that influence the mental state or behavior of a patient. The system can then display data characterizing anomalies in the patient's brain activity the user, so that the user is not forced to search through and analyze a large amount of data that is not clinically relevant. Therefore, the amount of time that a user must spend to discover the portion of the brain data that is useful can be drastically reduced, resulting in improved outcomes for patients, users and/or clinicians, especially when effective care requires time sensitive investigations.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIGS. 1A and 1B form a schematic block diagram of a computer system upon which arrangements described can be practiced.

FIG. 1C is a block diagram of an example mental state determination system.

FIGS. 2A and 2B show a window of a graphical user interface showing a graphical representation of a treatment plan in response to an anomaly detected in a subject.

FIG. 3 shows another window of a graphical user interface showing a template to submit a natural language input and generate a treatment plan.

FIG. 4 shows the window of FIG. 3 with a submitted natural language input.

FIG. 5 shows the window of FIG. 3 with a selected anomaly.

FIG. 6 is a flow diagram of an example method for determining a mental state of a patient that is of interest based on a natural language input and then determining whether a relevant subset of brain data is anomalous.

Like reference numbers and designations in the various drawings indicate like elements.

### DETAILED DESCRIPTION

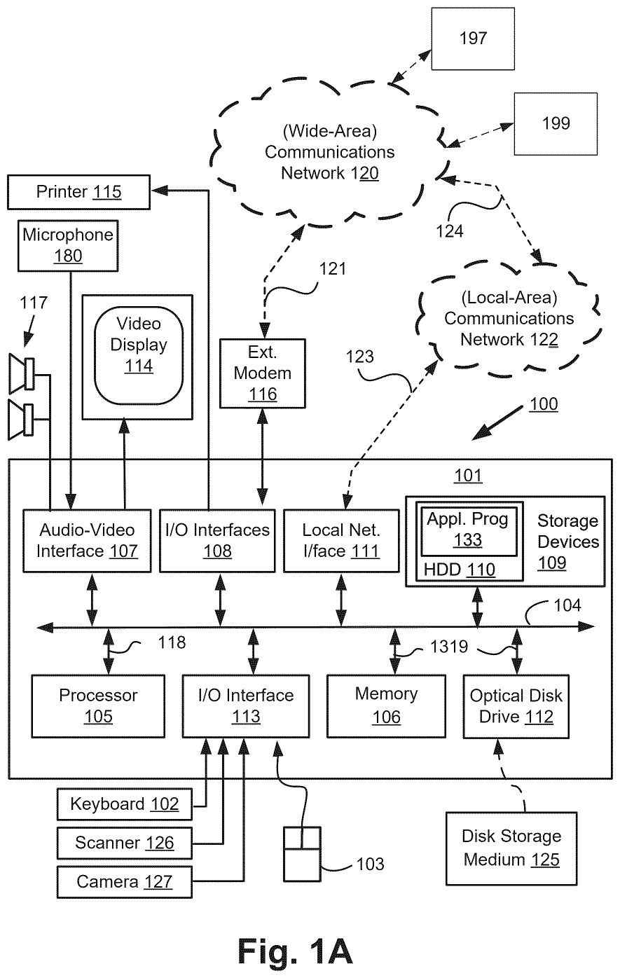

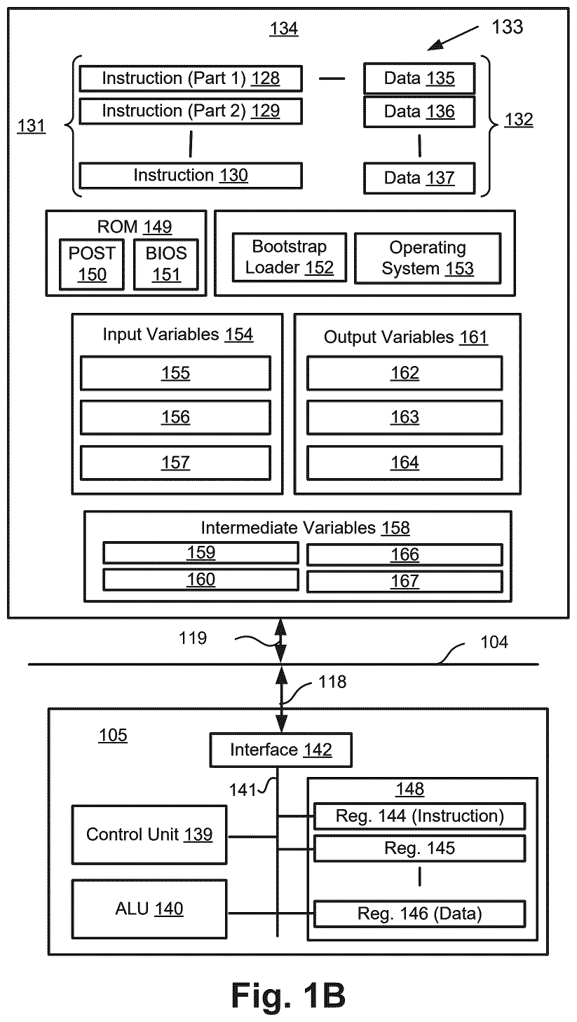

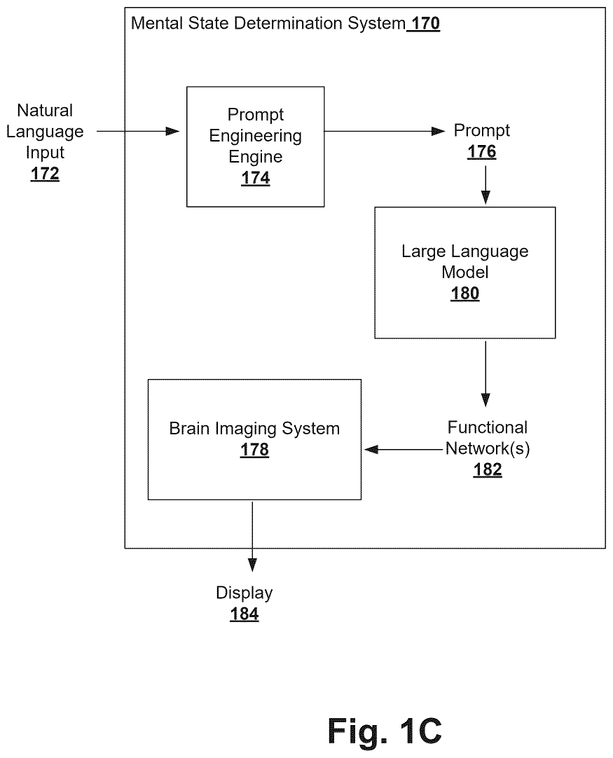

A computing device can perform the arrangements described. FIGS. 1A and 1B depict a computer system 100, upon which one can practice the various arrangements described.

As seen in FIG. 1A, the computer system 100 includes: a computer module 101; input devices such as a keyboard 102, a mouse pointer device 103, a scanner 126, a camera 127, and a microphone 180; and output devices including a printer 115, a display device 114 and loudspeakers 117. An external Modulator-Demodulator (Modem) transceiver device 116 may be used by the computer module 101 for communicating to and from a communications network 120 via a connection 121. The communications network 120 may be a wide-area network (WAN), such as the Internet, a cellular telecommunications network, or a private WAN. Where the connection 121 is a telephone line, the modem 116 may be a traditional “dial-up” modem. Alternatively, where the connection 121 is a high capacity (e.g., cable) connection, the modem 116 may be a broadband modem. A wireless modem may also be used for wireless connection to the communications network 120.

The computer module 101 typically includes at least one processor unit 105, and a memory unit 106. For example, the memory unit 106 may have semiconductor random access memory (RAM) and semiconductor read only memory (ROM). The computer module 101 also includes an number of input/output (I/O) interfaces including: an audio-video interface 107 that couples to the video display 114, loudspeakers 117 and microphone 180; an I/O interface 113 that couples to the keyboard 102, mouse 103, scanner 126, camera 127 and optionally a joystick or other human interface device (not illustrated); and an interface 108 for the external modem 116 and printer 115. In some implementations, the modem 116 may be incorporated within the computer module 101, for example within the interface 108. The computer module 101 also has a local network interface 111, which permits coupling of the computer system 100 via a connection 123 to a local-area communications network 122, known as a Local Area Network (LAN). As illustrated in FIG. 1A, the local communications network 122 may also couple to the wide network 120 via a connection 124, which would typically include a so-called “firewall” device or device of similar functionality. The local network interface 111 may comprise an Ethernet circuit card, a Bluetooth® wireless arrangement or an IEEE 802.11 wireless arrangement; however, numerous other types of interfaces may be practiced for the interface 111.

The module 101 can be connected with an image capture device 197 via the network 120. The device 197 can capture images of a subject brain using each of diffusor tension imaging and magnetic resonance imaging (MRI) techniques. The captured images are typically in standard formats such as DICOM format and OpenfMRI format respectively. The module 101 can receive DTI and MRI images the device 197 via the network 120. Alternatively, the DTI and MRI images can be received by the module 101 from a remote server, such as a cloud server 199, via the network 120. In other arrangements, the module 101 may be an integral part of one of the image capture device 197 and the server 199.

The I/O interfaces 108 and 113 may afford either or both of serial and parallel connectivity, the former typically being implemented according to the Universal Serial Bus (USB) standards and having corresponding USB connectors (not illustrated). Storage devices 109 are provided and typically include a hard disk drive (HDD) 110. Other storage devices such as a floppy disk drive and a magnetic tape drive (not illustrated) may also be used. An optical disk drive 112 is typically provided to act as a non-volatile source of data. Portable memory devices, such optical disks (e.g., CD-ROM, DVD, Blu ray Disc™), USB-RAM, portable, external hard drives, and floppy disks, for example, may be used as appropriate sources of data to the system 100.

The components 105 to 113 of the computer module 101 typically communicate via an interconnected bus 104 and in a manner that results in a conventional mode of operation of the computer system 100 known to those in the relevant art. For example, the processor 105 is coupled to the system bus 104 using a connection 118. Likewise, the memory 106 and optical disk drive 112 are coupled to the system bus 104 by connections 119. Examples of computers on which the described arrangements can be practised include IBM-PC's and compatibles, Sun Sparcstations, Apple Mac™ or like computer systems.

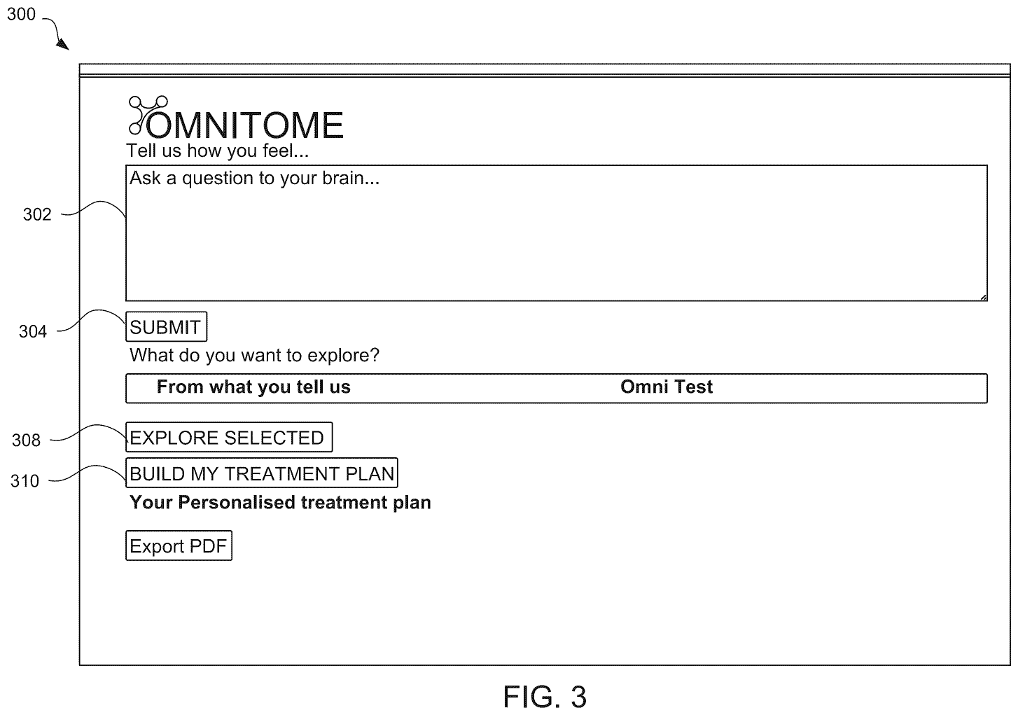

The method described may be implemented using the computer system 100 wherein the processes of FIGS. 3 to 5, to be described, may be implemented as one or more software application programs 133 executable within the computer system 100. In particular, the steps of the method described are effected by instructions 131 (see FIG. 1B) in the software 133 that are carried out within the computer system 100. The software instructions 131 may be formed as one or more code modules, each for performing one or more particular tasks. The software may also be divided into two separate parts, in which a first part and the corresponding code modules performs the described methods and a second part and the corresponding code modules manage a user interface between the first part and the user.

The software may be stored in a computer readable medium, including the storage devices described below, for example. The software is loaded into the computer system 100 from the computer readable medium, and then executed by the computer system 100. A computer readable medium having such software or computer program recorded on the computer readable medium is a computer program product. The use of the computer program product in the computer system 100 preferably effects an advantageous apparatus for providing a display of a neurological image.

The software 133 is typically stored in the HDD 110 or the memory 106. The software is loaded into the computer system 100 from a computer readable medium, and executed by the computer system 100. Thus, for example, the software 133 may be stored on an optically readable disk storage medium (e.g., CD-ROM) 125 that is read by the optical disk drive 112. A computer readable medium having such software or computer program recorded on it is a computer program product. The use of the computer program product in the computer system 100 preferably effects an apparatus for providing a display of a neurological image.

In some instances, the application programs 133 may be supplied to the user encoded on one or more CD-ROMs 125 and read via the corresponding drive 112, or alternatively may be read by the user from the networks 120 or 122. Still further, the software can also be loaded into the computer system 100 from other computer readable media. Computer readable storage media refers to any non-transitory tangible storage medium that provides recorded instructions and/or data to the computer system 100 for execution and/or processing. Examples of such storage media include floppy disks, magnetic tape, CD-ROM, DVD, Blu-Ray™ Disc, a hard disk drive, a ROM or integrated circuit, USB memory, a magneto-optical disk, or a computer readable card such as a PCMCIA card and the like, whether or not such devices are internal or external of the computer module 101. Examples of transitory or non-tangible computer readable transmission media that may also participate in the provision of software, application programs, instructions and/or data to the computer module 101 include radio or infra-red transmission channels as well as a network connection to another computer or networked device, and the Internet or Intranets including e-mail transmissions and information recorded on Websites and the like.

The second part of the application programs 133 and the corresponding code modules mentioned above may be executed to implement one or more graphical user interfaces (GUIs) to be rendered or otherwise represented upon the display 114. Through manipulation of typically the keyboard 102 and the mouse 103, a user of the computer system 100 and the application may manipulate the interface in a functionally adaptable manner to provide controlling commands and/or input to the applications associated with the GUI(s). Other forms of functionally adaptable user interfaces may also be implemented, such as an audio interface utilizing speech prompts output via the loudspeakers 117 and user voice commands input via the microphone 180.

FIG. 1B is a detailed schematic block diagram of the processor 105 and a “memory” 134. The memory 134 represents a logical aggregation of all the memory modules (including the HDD 109 and semiconductor memory 106) that can be accessed by the computer module 101 in FIG. 1A.

When the computer module 101 is initially powered up, a power-on self-test (POST) program 150 executes. The POST program 150 is typically stored in a ROM 149 of the semiconductor memory 106 of FIG. 1A. A hardware device such as the ROM 149 storing software is sometimes referred to as firmware. The POST program 150 examines hardware within the computer module 101 to ensure proper functioning and typically checks the processor 105, the memory 134 (109, 106), and a basic input-output systems software (BIOS) module 151, also typically stored in the ROM 149, for correct operation. Once the POST program 150 has run successfully, the BIOS 151 activates the hard disk drive 110 of FIG. 1A. Activation of the hard disk drive 110 causes a bootstrap loader program 152 that is resident on the hard disk drive 110 to execute via the processor 105. This loads an operating system 153 into the RAM memory 106, upon which the operating system 153 commences operation. The operating system 153 is a system level application, executable by the processor 105, to fulfil various high level functions, including processor management, memory management, device management, storage management, software application interface, and generic user interface.

The operating system 153 manages the memory 134 (109, 106) to ensure that each process or application running on the computer module 101 has sufficient memory in which to execute without colliding with memory allocated to another process. Furthermore, the different types of memory available in the system 100 of FIG. 1A must be used properly so that each process can run effectively. Accordingly, the aggregated memory 134 is not intended to illustrate how particular segments of memory are allocated (unless otherwise stated), but rather to provide a general view of the memory accessible by the computer system 100 and how such is used.

As shown in FIG. 1B, the processor 105 includes a number of functional modules including a control unit 139, an arithmetic logic unit (ALU) 140, and a local or internal memory 148, sometimes called a cache memory. The cache memory 148 typically includes a number of storage registers 144-146 in a register section. One or more internal busses 141 functionally interconnect these functional modules. The processor 105 typically also has one or more interfaces 142 for communicating with external devices via the system bus 104, using a connection 118. The memory 134 is coupled to the bus 104 using a connection 119.

The application program 133 includes a sequence of instructions 131 that may include conditional branch and loop instructions. The program 133 may also include data 132 which is used in execution of the program 133. The instructions 131 and the data 132 are stored in memory locations 128, 129, 130 and 135, 136, 137, respectively. Depending upon the relative size of the instructions 131 and the memory locations 128-130, a particular instruction may be stored in a single memory location as depicted by the instruction shown in the memory location 130. Alternately, an instruction may be segmented into a number of parts each of which is stored in a separate memory location, as depicted by the instruction segments shown in the memory locations 128 and 129.

In general, the processor 105 is given a set of instructions which are executed therein. The processor 105 waits for a subsequent input, to which the processor 105 reacts to by executing another set of instructions. Each input may be provided from one or more of a number of sources, including data generated by one or more of the input devices 102, 103, data received from an external source across one of the networks 120, 102, data retrieved from one of the storage devices 106, 109 or data retrieved from a storage medium 125 inserted into the corresponding reader 112, all depicted in FIG. 1A. The execution of a set of the instructions may in some cases result in output of data. Execution may also involve storing data or variables to the memory 134.

The described arrangements use input variables 154, which are stored in the memory 134 in corresponding memory locations 155, 156, 157. The described arrangements produce output variables 161, which are stored in the memory 134 in corresponding memory locations 162, 163, 164. Intermediate variables 158 may be stored in memory locations 159, 160, 166 and 167.

Referring to the processor 105 of FIG. 1B, the registers 144, 145, 146, the arithmetic logic unit (ALU) 140, and the control unit 139 work together to perform sequences of micro-operations needed to perform “fetch, decode, and execute” cycles for every instruction in the instruction set making up the program 133. Each fetch, decode, and execute cycle comprises:
a fetch operation, which fetches or reads an instruction 131 from a memory location 128, 129, 130;
a decode operation in which the control unit 139 determines which instruction has been fetched; and
an execute operation in which the control unit 139 and/or the ALU 140 execute the instruction.

Thereafter, a further fetch, decode, and execute cycle for the next instruction may be executed. Similarly, a store cycle may be performed by which the control unit 139 stores or writes a value to a memory location 132.

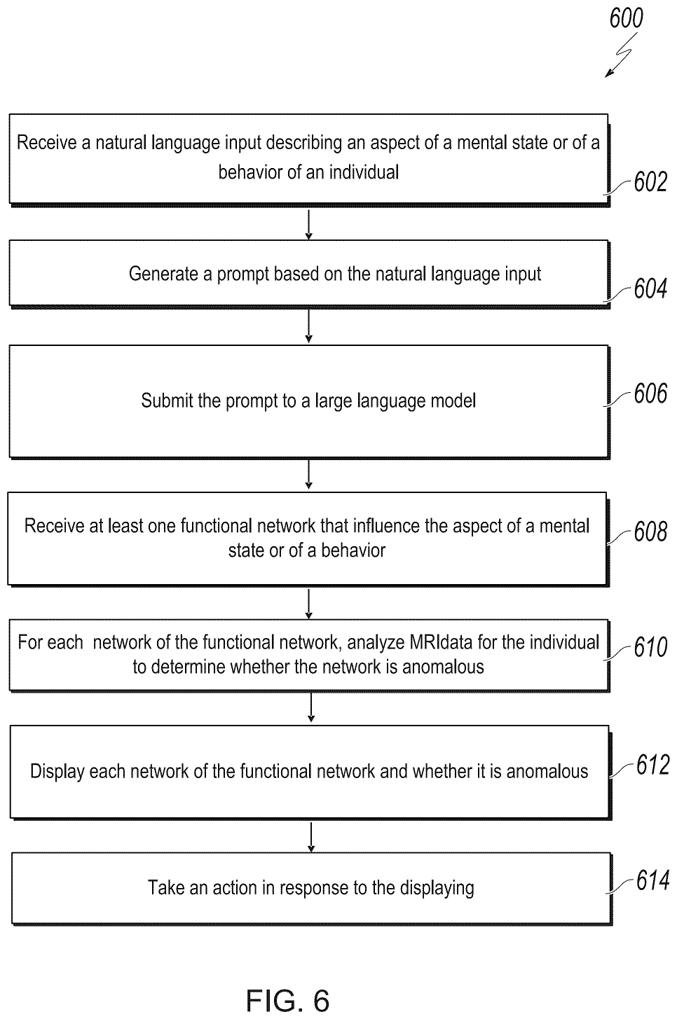

Each step or sub-process in the processes of FIG. 6 is associated with one or more segments of the program 133 and is performed by the register section 144, 145, 147, the ALU 140, and the control unit 139 in the processor 105 working together to perform the fetch, decode, and execute cycles for every instruction in the instruction set for the noted segments of the program 133.

FIG. 1C is a block diagram of an example mental state determination system 170.

The mental state determination system 170 includes a prompt engineering engine 174, a large language model 180, and a brain imaging system 178.

The prompt engineering engine 174 receives a natural language input 172 and generates a prompt 176. The natural language input 172 can describe at least one aspect of a mental state or of a behavior of an individual. The natural language input can be any description of one or more aspects of an individual's mental state or of an individual's behavior in any human language. For example, the natural language input can be a question or a statement. The prompt 176 can be any prompt that is configured to generate at least one functional network that influences the at least one aspect of a mental state or of a behavior above a threshold and can be submitted to a large language model. The prompt 176 can be based at least in part on the natural language input. Generating the prompt 176 is described in further detail below with reference to FIG. 3.

The large language model 180 receives the prompt 176 from the prompt engineering engine 174 and processes the prompt to generate one or more functional networks 182. The large language model 180 can be any appropriate neural network that receives an input sequence made up of text tokens selected from a vocabulary and auto-regressively generates an output sequence made up of text tokens from the vocabulary. For example, the large language model 180 can be a Transformer-based language model neural network or a recurrent neural network-based language model neural network.

The one or more functional networks 182 can be any functional networks that influence the at least one aspect of the mental state or of the behavior. Each functional network can include several smaller networks.

The brain imaging system 182 receives the one or more functional networks 182 from the large language model 180 and generates a display 184. For each network of the one or more functional networks, the brain imaging system 178 analyses MRI data for the individual to determine whether the network in anomalous. The network can be considered to be anomalous if the MRI data of the individual relevant to the network is outside a normal range, e.g., as defined by the brain data of other patients. The normal range can be, for example, defined by a standard deviation from the median of the distribution, defined by a range of values that cover a percentage (e.g., 95%) of the population, or defined by a distance from the mean of the distribution.

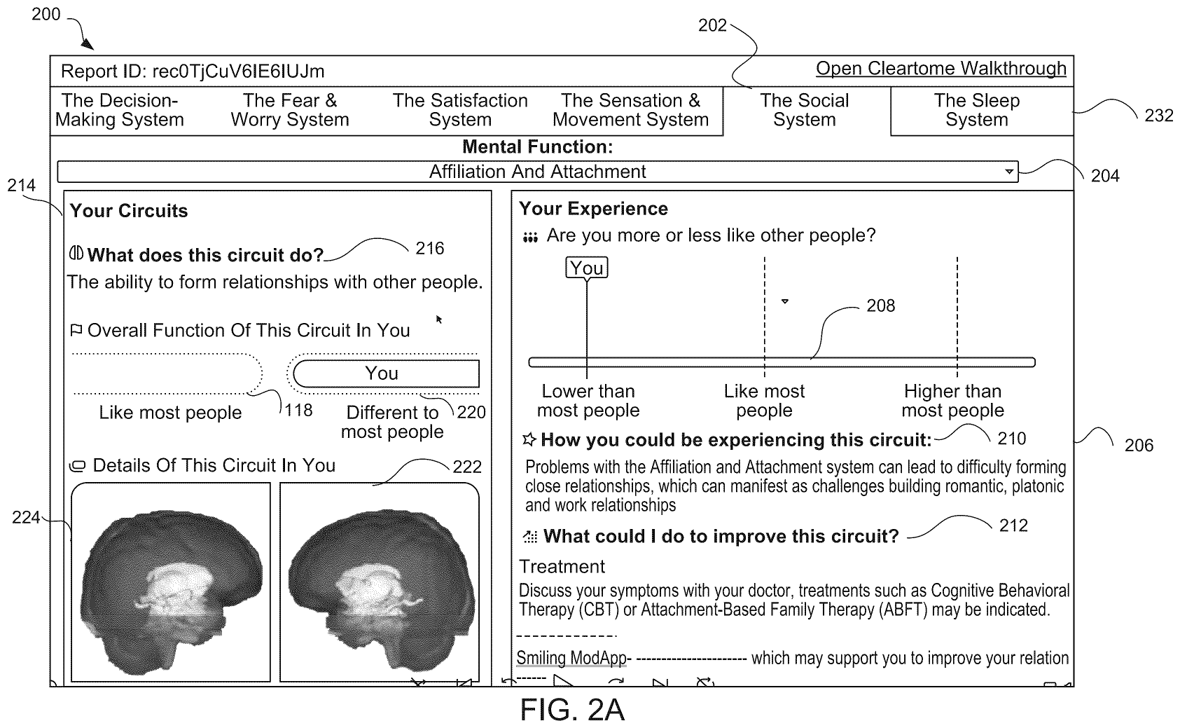

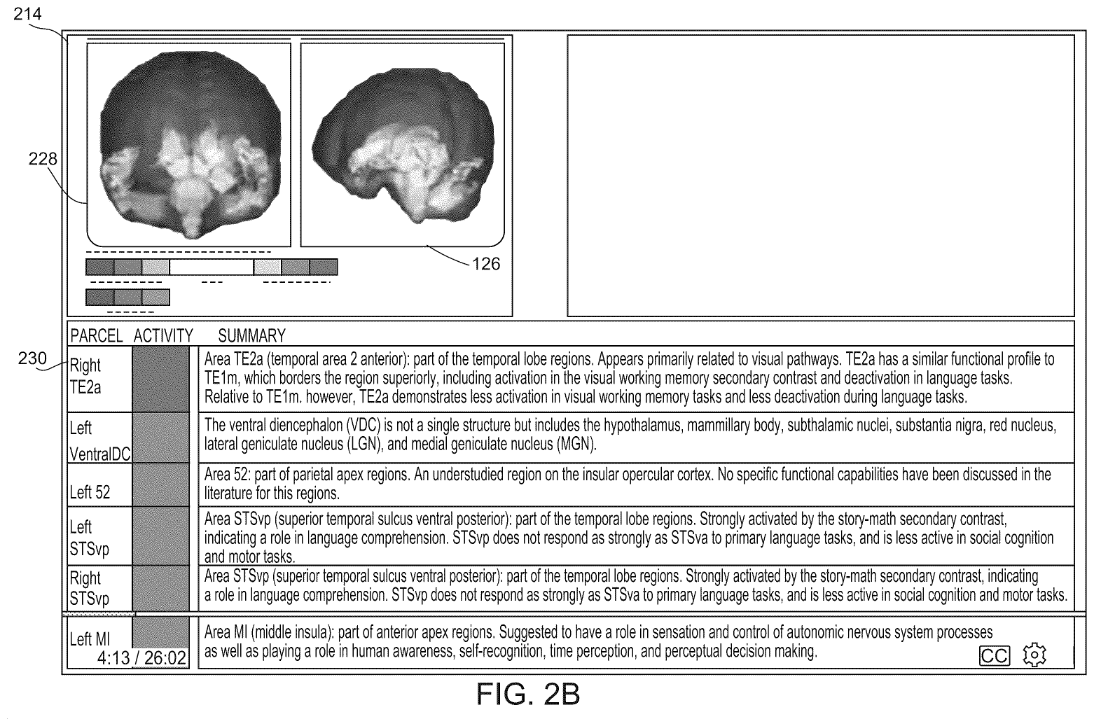

The display 184 can display each network of the at least one functional network and whether it is anomalous. The display can be a window of a graphical user interface such as the graphical user interfaces in FIGS. 2A-5.

FIGS. 2A and 2B show a graphical user interface showing a graphical representation of a treatment plan in response to an anomaly detected in a subject. A variety of techniques can be used for anomaly detection. For example, anomaly detection techniques described in U.S. Pat. No. 11,087,877, listing Michael Sughrue, Stephane Doyen and Peter Nicholas as inventors, entitled “identifying anomalous brain data” and incorporated herein by reference in its entirety, can be used for anomaly detection.

The window 200 includes six tabs 232 that each represent a system of the human brain. A user can select a tab to view details regarding the mental state or behavior of an individual associated with the system represented by the tab. The six tabs represent “The Decision-Making System”, “The Fear& Worry System”, “The Satisfaction System”, “The Sensation & Movement System”, “The Social System”, and “The Sleep System” as described in the research domain criteria (RDoC) framework. The RDoC framework organizes the human brain into different domains. In this example, a user has selected “The Social System” tab 202.

Each system breaks down further into a set of constructs associated with the system. A user can select a construct associated the with selected tab from a construct menu 204 to view details regarding the selected construct. For example, according to the RDoC Framework, the constructs associated with the “The Social System” tab 102 include “Affiliation and Attachment”, “Social Communication”, “Perception and Understanding of Self”, and “Perception and Understanding of Others”. In this example, a user has selected to view details regarding “Affiliation and Attachment”.

Once the user has selected a system from the six tabs 232 and a construct from the construct menu 204, the window displays an assessment 214 of brain circuits for the individual based on a subset of brain data for the individual regarding the selected construct. The brain data can be any data characterizing the brain of a patient. For example, brain data can include one or both of i) direct measurement data of the brain of the patient, e.g., images of the brain collected using brain imaging techniques, or ii) data that has been derived or generated from initial measurement data of the brain of the patient, e.g., correlation matrices.

The assessment 214 displays a description 216 of the brain circuit that is associated with the construct. In this example, the description 216 associated with the brain circuit for “Affiliation and Attachment” reads “The ability to form relationships with other people”.

The assessment 214 displays whether the subset of brain data for the individual indicates that the subset of brain data shows an anomaly 220 or does not show an anomaly 218. The subset of brain data is considered to show an anomaly, e.g., if a subset of brain data of the patient is outside a normal range, e.g., as defined by the brain data of other patients. The normal range can be, for example, defined by a standard deviation from the median of the distribution, defined by a range of values that cover a percentage (e.g., 95%) of the population, or defined by a distance from the mean of the distribution.

Additionally, with reference to FIGS. 2A and 2B, the assessment 214 displays anomaly data 222, 224, 226, 228, and 230 for the individual that shows activity levels for different portions of the brain. In this example, the assessment 214 displays four brain images 222, 224, 226, and 228 that show regions of abnormal activity as well as a table 230 that describes activity levels for different parcels of the brain as well as a summary for each parcel.

The brain images 222, 224, 226, and 228 are discretized using a brain map to reduce the brain data into smaller portion related to specific behaviors for the particular brain circuit e.g., the language region, the motion region, etc. The regions for which the quantity of activation is outside a normal range when compared across a proprietary database of brain data captured from hundreds, thousands, or millions of other individuals are highlighted in the assessment 214.

The table 230 organizes the individual's brain data according to multiple different parcellations of the individual's brain. For example, the brain data can include multiple different time series characterizing the activity of a respective different region of the brain of the individual over time, e.g., a time series corresponding to each three-dimensional voxel of the brain that can be measured by an MRI machine. The table 230 can organize the different time series signals by parcellation according to a brain atlas of the brain. A brain atlas is data that defines one or more parcellations of a brain of a patient, e.g., by defining in a common three-dimensional coordinate system the coordinates of the outline of the parcellation or the volume of the parcellation.

The window also displays an analysis 206 for the individual based on the individual's description 208 of their own mental state or behavior as well as a treatment plan 212 for the individual. In this example, the individual's description 208 indicates that they are less attached to other people than most people. The individual's description is a subjective description and can be obtained as a natural language description or as an answer to a survey.

The analysis 206 also displays symptoms 210 that the individual may experience as well as how the individual compares to others 208. In this example, symptoms for having lower affiliation and attachment with most people are described as “difficulty forming close relationships, which can manifest as challenges building romantic, platonic, and work relationships”.

The treatment plan 212 describes several possible actions that the individual can take to improve symptoms 210 that the individual may be experiencing 208. In this example, the treatment plan 212 suggests “treatments such as Cognitive Behavioral Therapy (CBT) or Attachment-Based Family Therapy (ABFT) may be indicated”.

A large language model generates the treatment plan 212. A large language model (“LLM”) is a model that is trained to process and generate human language. LLMs are trained on massive datasets of text and code, and they can be used for a variety of tasks. The large language model can be any appropriate neural network that receives an input sequence made up of text tokens selected from a vocabulary and auto-regressively generates an output sequence made up of text tokens from the vocabulary. For example, the large language model can be a Transformer-based language model neural network or a recurrent neural network-based language model neural network.

In some situations, the large language model can be referred to as an auto-regressive neural network when the neural network used to implement the large language model auto-regressively generates an output sequence of tokens. More specifically, the auto-regressively generated output is created by generating each particular token in the output sequence conditioned on a current input sequence that includes any tokens that precede the particular text token in the output sequence, i.e., the tokens that have already been generated for any previous positions in the output sequence that precede the particular position of the particular token, and a context input that provides context for the output sequence.

For example, the current input sequence when generating a token at any given position in the output sequence can include the input sequence and the tokens at any preceding positions that precede the given position in the output sequence. As a particular example, the current input sequence can include the input sequence followed by the tokens at any preceding positions that precede the given position in the output sequence. Optionally, the input and the current output sequence can be separated by one or more predetermined tokens within the current input sequence.

More specifically, to generate a particular token at a particular position within an output sequence, the large language model can process the current input sequence to generate a score distribution (e.g., a probability distribution) that assigns a respective score, e.g., a respective probability, to each token in the vocabulary of tokens. The large language model can then select, as the particular token, a token from the vocabulary using the score distribution.

As a particular example, the large language model can be an auto-regressive Transformer-based neural network that includes (i) a plurality of attention blocks that each apply a self-attention operation and (ii) an output subnetwork that processes an output of the last attention block to generate the score distribution.

The language model neural network 212 can have any of a variety of Transformer-based neural network architectures. The architectures can include OpenAI: ChatGPT, Falcon, Google Bard: LaMDA, Cohere or any of a variety of other large language models.

The large language model (e.g., when trained on a large dictionary database customized for brain data and/or when prompted appropriately) can generate a treatment plan in response to a request for how to address symptoms a user is experiencing and as verified by a brain data analysis system. A prompt generation system can generate a prompt from a dictionary database that includes, for each possible construct and level of reactivity (e.g., over-reactive, underactive), a list of symptoms and possible treatments for each symptom. A user can select symptom(s) to address and the prompt generation system can select a portion of the dictionary that is relevant to the selected system and parse the portion of the dictionary into a set of prompts. The large language model can process the set of prompts to generate the treatment plan 212.

FIG. 3 shows another window 300 of a graphical user interface showing a template to submit a natural language input and generate a treatment plan.

The window 300 includes a text box 302 to receive a natural language input from a user that describes one or more mental states or behaviors of an individual along with three buttons 304, 308 and 310 described below.

The natural language input can be any description of one or more aspects of an individual's mental state or behavior in any human language. For example, the natural language input can be a question or a statement. The submission button 304, when pressed, can result in generating a prompt that is based on the natural language input. The prompt can be configured to generate one or more functional networks that can influence the one or more aspects of the mental state or of the behavior.

When the natural language input is a question, the prompt can be a prompt to receive a section of a dictionary. The prompt can include instructions find one or more constructs that are related to the natural language input from a list of possible constructs e.g., action perception, action planning, acute threat, affiliation attachment, agency ownership, etc. The prompt can include instructions to use the natural language input to receive a section of a customized dictionary relevant to the one or more relevant constructs.

The dictionary can define, for each construct, symptoms for “overactive” networks and “underactive” networks. For each symptom, the dictionary can define possible treatments. For example, when the construct is Affiliation and Attachment and the natural language prompt may indicate an overactive network, the dictionary can define “Clinginess” as a symptom and “CBT”, “Dialectical behavior therapy”, and “Group therapy” as treatments for clinginess.

An example dictionary is shown below:

```
rdoc_constructs_symtoms_treatments = {
  “Adjusting Expectations” : {
      “overactive” : {
          “Unrealistic expectations” : [“CBT”, “Mindfulness techniques”,
“Psychoeducation”] ,
          “Perfectionism”: [“CBT”, “Mindfulness techniques”, “Acceptance and
Commitment Therapy (Act)”]
      } ,
      “underactive”: {
          “Low expectations” : [“CBT”, “Behavioral activation”, “Goal-setting”] ,
          “Difficulty adapting to change”: [“CBT”, “Mindfulness techniques”, “See a
healthcare professional”]
      }
  } ,
  “Affiliation and Attachment” : {
      “overactive”: {
          “Clinginess” : [“CBT”, “Dialectical behavior therapy”, “Group therapy”] ,
          “Fear of abandonment” : [“CBT”, “Behavioral activation”, “Group therapy”]
      } ,
      “underactive” : {
          “Difficulty forming attachments”: [“CBT”, “IPT”, “See a healthcare
professional”]
      }
  } ,
  “Anticipation” : {
      “overactive” : {
          “Excessive worry” : [“CBT”, “Mindfulness techniques”, “Medication”] ,
          “Fear of future events” : [“CBT” “Exposure therapy”, “Medication”]
    } ,
    “underactive” : {
        “Lack of motivation” : [“CBT”, “Behavioral activation”, “See healthcare
professional”] ,
        “Difficulty planning for future” : [“CBT”, “Goal-setting”, “Time
management”]
    }
  } ,
  “Arousal” : {
    “overactive” : {
          “Hyperarousal”: [“CBT”, “Relaxation techniques”, “Medication”] ,
          “Anxiety” : [“CBT”, “Mindfulness techniques”, “Medication”]
    } ,
    “underactive” : {
          “Lethargy” : [“Behavioral activation”, “Physical activity”, “See a healthcare
professional”] ,
          “Difficulty staying awake” : [“Sleep hygiene”, “Medication”, “See a
healthcare professional”]
    }
```

The prompt can use the natural language input to query the dictionary. The prompt can include instructions to process the natural language input to determine if the symptoms described in the natural language input describe an underactive or overactive network relevant to one or more constructs and then return the section of the dictionary that is relevant to that construct. For example, if the natural language input is “Why is it difficult for me to be away from my friends when I'm at work?”, the prompt can query the dictionary to find that clinginess is a symptom of an overreactive Affiliation and Attachment construct. The large language model can return the section of the dictionary that is relevant to Affiliation and Attachment. An example prompt is shown below:

```
question_to_dict(user_input) :
return [
  {“role”: “system” , “content”: “You will only provide answers in the shape of a
  python dictionary. No additional text, just the python dictionary.”} ,
  {“role”: “user”, “content”: “Do you know RDDC framework for mental health?”} ,
  {“role”: “assistant”, “content”: “Yes, I'm familiar with the Research Domain Criteria
  (RDoC) framework. RDoC is a research framework developed by the National
  Institute of Mental Health
  {“role”: “user” , “content”: “”What are all the potential RDOC constructs only related
  to the question in the next prompt. You will only provide short answers in the form of
  a python dictionary
  Find in that sentence whether the construct is likely to be overactive or underactive
  and use this to build in the dictionary mentioned further.
```

The dictionary should follow the very strict structure as follows:

```
{{ ‘overactive’:
  ‘construct_1’
  ‘construct_2’
  etc.
  ],
‘underactive’: [
  ‘construct_1’
  ‘construct_2’
  etc.
  ]
}}
Constructs can only be drawn from that list:
  action perception
  action planning
  acute threat
  affiliation attachment
  agency ownership
  arousal
  attention
  circadian rhythm
  cognitive inhibition
  declarative memory
  effort
  frustrative nonreward
  goal selection
  habit PVS
  head injury
  headache
  interoception
  language
  loss grief
  others harm
  performance monitoring
  potential threat
  production facial communication
  reward anticipation
  reward delayed
  reward prediction error
  reward probability
  reward receipt
  reward satiation
  self agency
  selft harm
  self knowledge
  sleep wake
  sustained threat
  vistual perception
  wm active maintenance
  wm capacity
  wm flexible updating
  wm interference control
Limit your answer to only those that are relevant to the questions below
’”
{“role”: “user”, “content” : “Question: ‘{question }’”. format(question=user_input)}].
```

The window also includes an exploration button 308 to view more details regarding a functional network (e.g., brain images for the individual that highlight portions of the individual's brain that show abnormal activity) and a treatment plan button 310 to generate possible treatments in response to the mental state or the behavior of the individual.

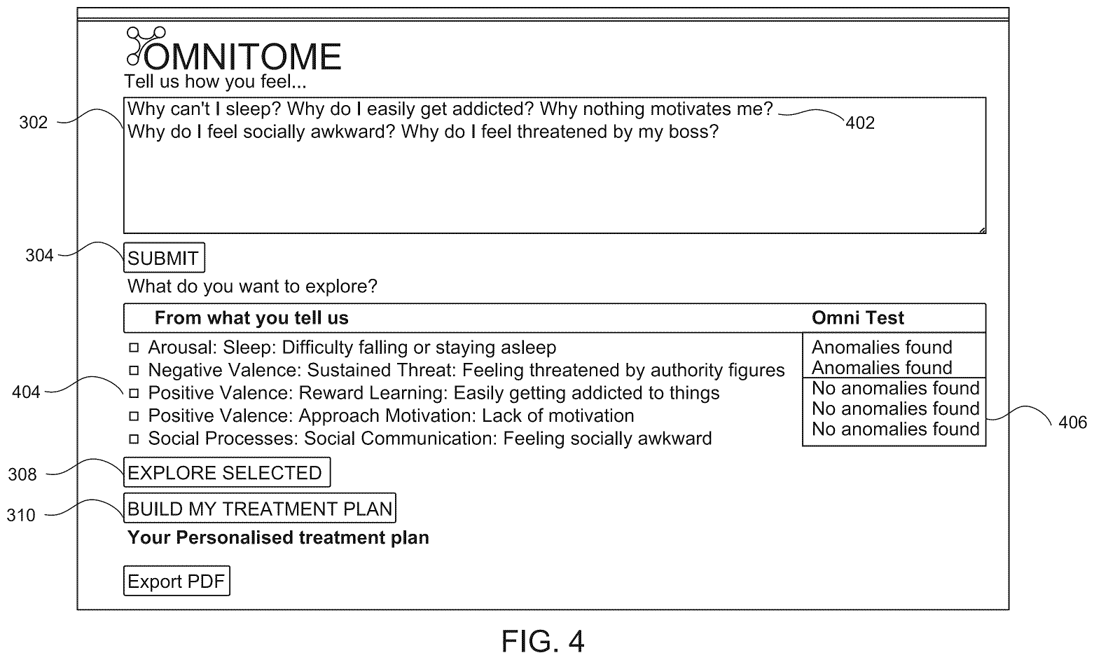

FIG. 4 shows the window of FIG. 3 with a submitted natural language input. The text box 202 receives a natural language input “Why can't I sleep? Why do I easily get addicted? Why nothing motivates me? Why do I feel socially awkward? Why do I feel threatened by my boss?” that describes multiple aspects of a mental state of a user.

In some examples, the natural language input can describe one or more aspects of a behavior of an individual e.g., “Others don't think I'm fun to be around”.

A prompt generation system can generate a prompt based on the natural language that is configured to generate at least one functional network that influences the at least one aspect of a mental state or a behavior above a threshold.

A large language model can process the prompt to generate the one or more functional networks that can influence one or more aspects of the mental state or of the behavior. Each functional network can include several smaller networks.

The window displays a summary 404 that describes symptoms associated with the network anomalies. For example, a summary can read “Arousal: Sleep: Difficulty falling or staying asleep”. In this example, the large language model generates five sub-symptoms that may influence the mental state or behavior.

For each sub-symptom, an anomaly detection system analyzes MRI data for the individual to determine whether the network(s) correlated with the sub-symptom is anomalous in that particular individual. The window includes an anomaly display 306 that identifies whether each network is anomalous or not for the individual.

A user can take an action in response to the anomaly display 406. For example, when a user selects the treatment plan button 310, the user can select one or more functional networks and/or sub-symptoms to explore further. A large language model can receive a request for how to address an anomaly in the selected functional network and generate a relevant treatment plan.

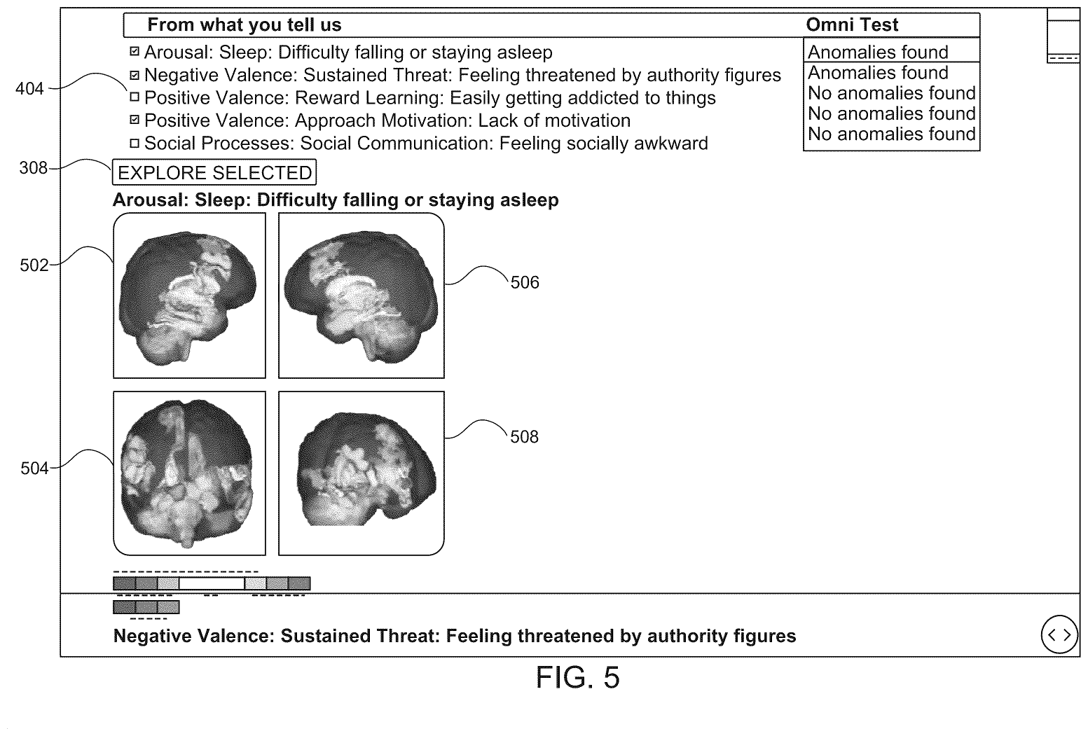

FIG. 5 shows the window of FIG. 3 with a selected anomaly. In this example, the summary 404 of a selected anomaly reads “Arousal: Sleep: Difficulty falling or staying asleep”. When a user selects exploration button 308, the window can display brain images for the individual 502, 504, 506, and 508 that highlight portions of the individual's brain that show abnormal activity.

FIG. 6 is a flow diagram of an example method 600 for determining a mental state or a behavior of a patient that is of interest based on a natural language input and then determining whether a relevant subset of brain data is anomalous. For convenience, the method 600 will be described as being performed by a system, such as the system illustrated in FIG. 1A.

The system can receive a natural language input describing at least one aspect of a mental state or of a behavior of an individual (step 602). The natural language input can be a question. For example, the prompt can be “Why am I so sad?” when the prompt describes an aspect of a mental state. As another example, the prompt can be “Why do others think I'm not fun to be around?” when the prompt describes an aspect of a behavior.

The system can generate a prompt based at least in part the natural language input (step 604). The prompt can be configured to generate at least one functional network that influences the at least one aspect of a mental state or of a behavior above a threshold.

The system can submit the prompt to a large language model (step 604). The large language model can be any appropriate neural network that receives an input sequence made up of text tokens selected from a vocabulary and auto-regressively generates an output sequence made up of text tokens from the vocabulary. For example, the large language model can be a Transformer-based language model neural network or a recurrent neural network-based language model neural network.

The system can receive at least one functional network from the large language model that influences the at least one aspect of a mental state or of a behavior (step 606).

The system can, for each network of the at least one functional network, analyze MRI data for the individual to determine whether the network is anomalous (step 608). The network is considered to be anomalous if a MRI data regarding the network is outside a normal range, e.g., as defined by the brain data of other patients. The normal range can be, for example, defined by a standard deviation from the median of the distribution, defined by a range of values that cover a percentage (e.g., 95%) of the population, or defined by a distance from the mean of the distribution.

The system can display to the user each network of the at least one functional network and whether it is anomalous (step 610).

In some implementations, the system can receive a user input in response to the displaying. The response can include the selection of at least one functional network to produce at least one selected functional network.

The system can take an action in response to the displaying (step 612).

In some implementations, taking an action includes submitting a request for how to address an anomaly in the at least one selected functional network to a large language model. The large language model can generate one or more treatment plans for how to address the anomaly in the at least one selected functional network. The treatment plan can include surgeries, behavioral therapies, medical treatments (e.g., medications), brain editing techniques (e.g., brain stimulation, pharmacology, etc.), and at-home treatments.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A computer-implemented method comprising:
receiving a natural language input describing at least one aspect of a mental state or of a behavior of an individual;
using a prompt engineering engine, generating a prompt based on at least in part the natural language input, the prompt configured to generate at least one functional network that influences the at least one aspect of a mental state or of a behavior above a threshold;
submitting the prompt to a large language model, the prompt prompting the large language model to generate at least one functional network that influences the at least one aspect of a mental state or of a behavior;
receiving, from the large language model, the at least one functional network that influences the at least one aspect of a mental state or of a behavior;
for each network of the at least one functional network, analyzing MRI data for the individual to determine whether the network is anomalous, wherein analyzing MRI data for the individual comprises:
obtaining brain data captured by one or more sensors characterizing brain activity of the individual;
for each of a plurality of pairs of parcellations formed from a set of parcellations where each pair comprises a first parcellation and a second parcellation, processing the brain data to generate first connectivity data comprising a correlation between the brain activity of the first parcellation and the brain activity of the second parcellation in the brain of the patient, wherein the plurality of pairs of parcellations comprises at least 45 pairs of parcellations;
obtaining second connectivity data that characterizes, for each of the plurality of pairs of parcellations, a normal range of correlations between the brain activity of the first parcellation and the brain activity of the second parcellation of the pair of parcellations; and
identifying, using an anomaly detection system, one or more of the plurality of pairs of parcellations for which the correlation between brain activity of the first parcellation and the brain activity of the second parcellation of the pair is outside of a corresponding normal range of correlation specified in the second connectivity data;
displaying to a user each network of the at least one functional network and whether it is anomalous; and
taking an action in response to the displaying.

2. The method of claim 1, wherein the method further comprises receiving a user input in response to the displaying, wherein the response includes selection of at least one functional network to produce at least one selected functional network; and taking an action comprises submitting to a second large language model a request for how to address an anomaly in the at least one selected functional network.

3. The method of claim 2, wherein taking an action further comprises generating a plurality of treatment plans for how to address the anomaly in the at least one selected functional network.

4. The method of claim 3, wherein a treatment plan comprises (i) surgeries, (ii) behavioral therapies, (iii) brain editing techniques, or a combination thereof.

5. The method of claim 1, wherein the natural language input comprises one or more questions.

6. The method of claim 1, wherein the method further comprises in response to submitting the prompt to the large language model, receiving a mental state or a behavior.

7. The method of claim 6, wherein the method further comprises displaying the mental state or behavior along with an indication of whether that mental state or behavior is anomalous.

8. (canceled)

9. (canceled)

10. (canceled)

11. (canceled)

12. (canceled)

13. (canceled)

14. A system comprising:
a user device; and
one or more computers configured to interact with the user device and to perform operations comprising:
receiving a natural language input describing at least one aspect of a mental state or of a behavior of an individual;
using a prompt engineering engine, generating a prompt based on at least in part the natural language input, the prompt configured to generate at least one functional network that influences the at least one aspect of a mental state or of a behavior above a threshold;
submitting the prompt to a first large language model, the prompt prompting the large language model to generate at least one functional network that influences the at least one aspect of a mental state or of a behavior;
receiving, from the large language model, the at least one functional network that influences the at least one aspect of a mental state or of a behavior;
for each network of the at least one functional network, analyzing MRI data for the individual to determine whether the network is anomalous, wherein analyzing MRI data for the individual comprises:
obtaining brain data captured by one or more sensors characterizing brain activity of the individual;
for each of a plurality of pairs of parcellations formed from a set of parcellations where each pair comprises a first parcellation and a second parcellation, processing the brain data to generate first connectivity data comprising a correlation between the brain activity of the first parcellation and the brain activity of the second parcellation in the brain of the patient, wherein the plurality of pairs of parcellations comprises at least 45 pairs of parcellations;
obtaining second connectivity data that characterizes, for each of the plurality of pairs of parcellations, a normal range of correlations between the brain activity of the first parcellation and the brain activity of the second parcellation of the pair of parcellations; and
identifying, using an anomaly detection system, one or more of the plurality of pairs of parcellations for which the correlation between brain activity of the first parcellation and the brain activity of the second parcellation of the pair is outside of a corresponding normal range of correlation specified in the second connectivity data;
displaying to a user each network of the at least one functional network and whether it is anomalous; and
taking an action in response to the displaying.

15. The system of claim 14, wherein the operations further comprise receiving a user input in response to the displaying, wherein the response includes selection of at least one functional network to produce at least one selected functional network; and taking an action comprises submitting to a second large language model a request for how to address an anomaly in the at least one selected functional network.

16. The system of claim 15, wherein taking an action further comprises generating a plurality of treatment plans for how to address the anomaly in the at least one selected functional network.

17. The system of claim 16, wherein a treatment plan comprises (i) surgeries, (ii) behavioral therapies, (iii) brain editing techniques, or a combination thereof.

18. The system of claim 14, wherein the natural language input comprises one or more questions.

19. The system of claim 14, wherein the operations further comprise in response to submitting the prompt to the large language model, receiving a mental state or a behavior.

20. The system of claim 14, wherein the user device comprises a personal computer or smart phone running a web browser or a mobile telephone running a WAP browser.

21. The system of claim 15, wherein the second large language model is the same as the first large language model.
